(19) United States
(12) Patent Application Publication
(10) Pub. No.: US 2012/0103347 A1
(43) Pub. Date: May 3, 2012

(54) URINARY INCONTINENCE DEVICE AND METHOD

(75) Inventors: Amy Wheaton, Lindenhurst, IL (US); Loren Bartz, Brookfield, WI (US); Terence King, Lindenhurst, IL (US); Thom Nichols, Mequon, WI (US); George J. Cisko, JR., Spring Grove, IL (US); Jason Merryman, Grayslake, IL (US); Richard Murahata, Libestyville, IL (US); Robin RAy Thompson, Pleasant prairie, WI (US); Vit Ulinskas, Marina Del Rey, CA (US); Sona Patadia, Chicago, IL (US); Carolyn Rose, Chicago, IL (US)

(73) Assignee: HOLLISTER INCORPORATED, Libertyville, IL (US)

(21) Appl. No.: 13/322,685

(22) PCT Filed: May 4, 2010
(86) PCT No.: PCT/US10/33528

§ 371 Date: Dec. 23, 2011

Related U.S. Application Data
(62) Division of application No. 61/220,753, filed on Jun. 26, 2009.

(51) Int. Cl. A61F 5/455; A61F 5/453

(52) U.S. Cl. 128/885

(57) ABSTRACT

A urinary incontinence device comprising a flexible but shape-retaining body portion is disclosed wherein the body portion has a longitudinal axis extending from an anterior end to a posterior end thereof. The body portion is elongated in the direction of the longitudinal axis and has a length and width sufficient to cover the vulva or retracted penis of a human female or male and to form a urine-receiving receptacle. The receptacle h a shallow urine directing portion sloping inwardly and downwardly away from the vulva or retracted penis, and a deeper, narrower central urine collection portion having a urine outlet. The receptacle is elongated in the direction of the longitudinal ax of the body portion. The device also includes an attachment flange having an anterior end and a posterior end corresponding to the anterior end and the posterior end of the body portion, respectively. The attachment flange is formed to extend outwardly away from the body portion, and it includes an adhesive thereon for securing the device to entirely cover the vulva or retracted penis of a human female or male.

### FIELD OF THE DISCLOSURE

The present disclosure relates to devices and methods for use by those having occasional or chronic urinary incontinence problems and, more particularly, to such a device and method for urinary incontinence that is not only discrete and comfortable but also highly effective.

### BACKGROUND OF THE DISCLOSURE

Urinary incontinence is a well-known problem that has been addressed in a wide variety of ways for both males and females. However, the acceptable management of urinary incontinence in females, and males having retracted penises, has generally been viewed as much more difficult to achieve due primarily to the anatomy of the female external urogenital organs and the anatomy presented in a male having a retracted penis. As a result, it is generally agreed there has yet to be a device for these applications that is entirely acceptable in every respect.

In order to be entirely acceptable, a urinary incontinence device should not only be highly effective in preventing any leakage of urine, but it should also be discrete and comfortable when it is being used. These competing concerns have not been successfully addressed because it at least initially appears that no single device could possibly be provided to address all of these requirements while also being capable of use for an extended period of time. For instance, many urinary incontinence devices proposed for females have, at best, been found to either be ineffective in preventing urine leakage or lacking in discreteness due to their size and shape.

With regard to incontinent males having retracted penises, it is usually anatomically impossible for them to use conventional male external catheters that fit over and adhere to the penis, and there have been only a few commercially available devices which specifically address the anatomical problem in providing a urinary incontinence device for a male having a retracted penis that is discrete and comfortable while also being capable of preventing the leakage of urine

In addition to these problems, many female urinary incontinence devices have been lacking in comfort during use. This has rendered such devices less than entirely desirable for those suffering from urinary incontinence who ideally want an effective, discrete and comfortable device that can be used for a minimum of 24 hours. Further, many female urinary incontinence devices are such that they are incapable of fitting a variety of sizes.

An additional problem encountered by females suffering from urinary incontinence is the absence of any reliable way to easily and effectively position the device. It is common for many proposed female urinary incontinence devices to fit over the external genitalia and be affixed to the skin with an adhesive barrier material so it is important for the device to be properly placed for both comfort and function. Proper placement is particularly important in the perineum where the device must adhere securely and do so without interfering with bowel movements.

For many users of female urinary incontinence devices, proper placement requires that the device be applied by a caregiver. It is usually quite difficult for the users of such devices to be able to accurately locate the appropriate anatomical markers and then to be able to appropriately apply the device in the desired position for use. This is particularly true for overweight or obese users or those having impaired vision or reduced manual dexterity.

Moreover, it is generally well known that women, especially in the older population, are sometimes reluctant to view and touch the perineum and anus. Also, it is difficult, or at the very least inconvenient, to have available and/or use mirrors and gloves for self-application of female urinary incontinence devices. However, there has yet to be a truly acceptable method for a user to easily and effectively position a female urinary incontinence device without a caregiver.

For females suffering from varying types of incontinence, including stress, urge, and mixed incontinence, the absence of any entirely satisfactory female urinary incontinence device erodes their quality of life. This is true for women in nursing homes and hospitals who may be relatively immobile or bedridden, but it is also particularly true for ambulatory women who might otherwise be able to lead a relatively normal life. For ambulatory women, the inability to successfully address urinary incontinence can seriously impair their quality of life leading to other medical, emotional and social problems often accompanying incontinence.

In view of the foregoing, there has been a continuing need for a female urinary incontinence device for use by both immobile and ambulatory women that overcomes the problems with existing devices by preventing leakage while being discrete and comfortable when being used even over an extended period of time and regardless of size.

### SUMMARY OF THE DISCLOSURE

Accordingly, a urinary incontinence device comprising a flexible but shape-retaining body portion is disclosed wherein the body portion has a longitudinal axis extending from an anterior to a posterior end. The body portion is elongated in the direction of the longitudinal axis and has a length and width sufficient to comprise a urine-receiving receptacle when the device is placed on a human female or male in a position for use. The receptacle has a shallow urine directing portion sloping inwardly and downwardly away from the body, and a deeper, narrower central urine collection portion having a urine outlet. The receptacle is elongated in the direction of the longitudinal axis of the body portion. The urinary incontinence device also includes an attachment flange which is formed to have an anterior and a posterior end which correspond to the anterior and posterior end of the body portion, respectively. The attachment flange is formed to extend outwardly away from the body portion, and it includes an adhesive thereon for securing the device to the body between the legs of either a human female or male.

In an exemplary embodiment, the elongated body portion is curvilinear at the posterior end and has generally parallel sides that flare laterally outwardly to have a maximum width at the anterior end thereof. The central urine collection portion also has an anterior end and a posterior end, and the urine outlet is located proximate the posterior end thereof. Preferably, the shallow urine directing portion of the receptacle includes a vent which is located proximate the anterior end of the body portion to facilitate drainage of urine through the urine outlet.

In addition, the attachment flange extending outwardly away from the body portion advantageously comprises a flat, flexible body-facing surface having the adhesive thereon and extending entirely about the perimeter of the body portion. The attachment flange may also include a pair of anterior attachment tabs and a pair of posterior attachment tabs each integrally associated with the attachment flange. Preferably, each of the attachments tabs has an adhesive thereon and extends away from the body portion on opposite sides of the longitudinal axis to assist the attachment flange in securing the device to a human female or male.

Further, the urine outlet may advantageously include a flexible tubular fitting extending downwardly and rearwardly away from the central urine collection portion for connection to a urine drain tube and drainage bag. The device may also include a release sheet for protectively covering the adhesive on the attachment flange where the release sheet can be removed to expose the adhesive for securing the device to a human female or male. In one embodiment, the release sheet may comprise a split sheet having one sheet portion overlapping the other sheet portion to facilitate removal of the release sheet prior to use of the device.

Additionally, the device may include an area of weaker adhesive strength on the anterior end of the attachment flange and an area of stronger adhesive strength on the remainder of the attachment flange. Preferably, the area of weaker adhesive strength will extend from the anterior end toward the posterior end of the attachment flange at least as far as the anterior end of the body portion since the anterior end will be located in the pubic region. With this arrangement, the area of stronger adhesive strength will extend on the remainder of the attachment flange from the area of weaker adhesive strength to the posterior end of the attachment flange.

Furthermore, the attachment flange may advantageously extend outwardly to entirely surround the body portion while also extending inwardly over the posterior end of the body portion located posterior to the urine outlet to minimize urine contact with the skin.

A method for positioning a urinary incontinence device is also disclosed comprising the step of providing a flexible but shape-retaining body portion having an anterior end and a posterior end. The body portion of the device defines a receptacle for receiving urine which is surrounded by a flange having an adhesive for securing the device to a human female or male in a position for use. The method also includes the step of providing a finger shield on the posterior end of the body portion externally of the urine-receiving receptacle for use in positioning the device in a position for use. The method still further includes the step of placing a finger in the finger shield and positioning the device with the urine-receiving receptacle facing the vulva or retracted penis of a human female or male. The method additionally includes the step of locating the anus with the finger in the finger shield before the adhesive on the flange makes substantial contact with the skin of the human female or male. Further, the method includes the step of applying pressure to the flange to cause the adhesive to make substantial contact with the skin to secure the device to the human female or male in a position for use.

In one embodiment, the method may include the step of removing the finger shield from the posterior end of the body portion after the device has been secured to a human female or male in a position for use. The method may also include the flange having a posterior end and the finger shield extending approximately 1 inch beyond the posterior end of the flange. In still another embodiment, the method may include the body portion having a longitudinal axis and the finger shield being located to lie along the longitudinal axis of the body portion.

Other advantages and features of the present disclosure will become apparent from a consideration of the following specification taken in conjunction with the accompanying drawings.

### BRIEF DESCRIPTION OF THE DRAWINGS

FIG. 1 is a bottom perspective view of a urinary incontinence device in accordance with the present disclosure;

FIG. 2 is a side elevational view of the urinary incontinence device in accordance with the present disclosure;

FIG. 3 is a rear elevational view of the urinary incontinence device in accordance with the present disclosure;

FIG. 4 is a bottom elevational view of the urinary incontinence device in accordance with the present disclosure;

FIG. 5 is a top elevational view of the urinary incontinence device in accordance with the present disclosure;

FIG. 6 is a top elevational view of the urinary incontinence device having absorptive foam on a portion of the attachment flange;

FIG. 6A is across-sectional view showing material details for the urinary incontinence device generally at 6A-6A of FIG. 6;

FIG. 6B is a cross-sectional view showing material details for the urinary incontinence device generally at 6B-6B of FIG. 6;

FIG. 7 is a top elevational view of the urinary incontinence device having absorptive foam on substantially all of the attachment flange;

FIG. 7A is across-sectional view showing material details for the urinary incontinence device generally at 7A-7A of FIG. 7;

FIG. 7B is a cross-sectional view showing material details for the urinary incontinence device generally at 7B-7B of FIG. 7;

FIG. 8A is a bottom elevational view of the urinary incontinence device of FIG. 1 having a finger shield to assist in positioning the device; and

FIG. 8B is a bottom elevational view of the urinary incontinence device of FIG. 8A illustrating a finger within the finger shield.

### DETAILED DESCRIPTION OF THE DISCLOSURE

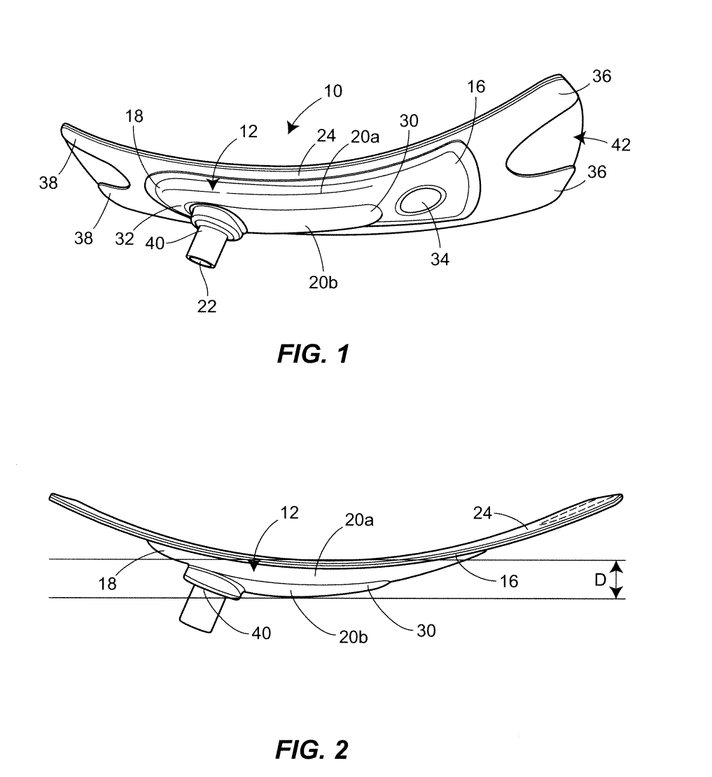

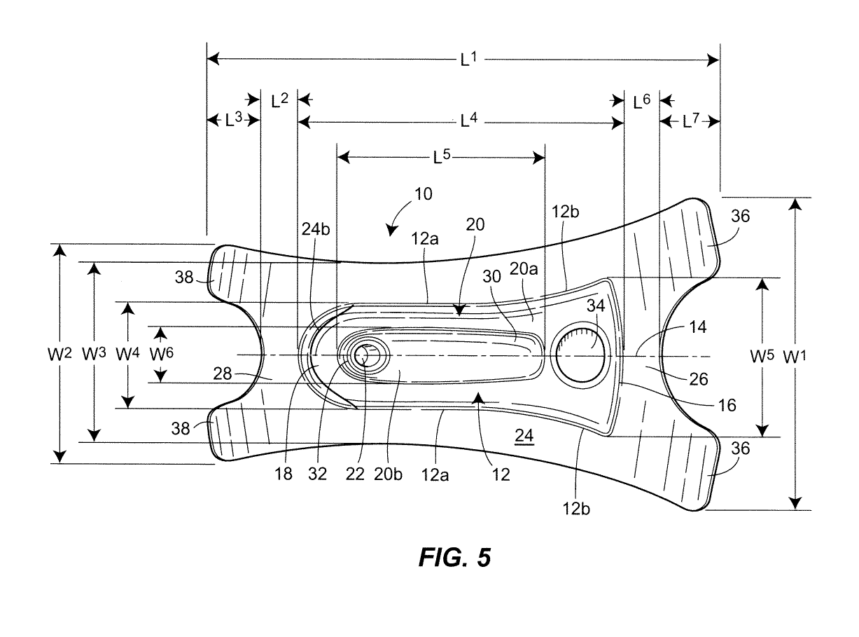

In the illustrations given, and with reference first to FIG. 1, the reference numeral 10 will be understood to designate generally a urinary incontinence device in accordance with the present disclosure. The device 10 comprises a flexible but shape-retaining body portion generally designated 12 having a longitudinal axis 14 extending from an anterior end 16 to a posterior end 18 thereof (FIG. 5). The body portion 12 is elongated in the direction of the longitudinal axis 14 and has a length and width sufficient to comprise a urine-receiving receptacle 20 when in a position for use. The receptacle 20 has a shallow urine directing portion 20a sloping inwardly and downwardly away from the body, and a deeper, narrower central urine collection portion 20b having a urine outlet as at 22. The receptacle 20 is elongated in the direction of the longitudinal axis 14 of the body portion 12. The device 10 also includes an attachment flange 24 having an anterior end 26 and a posterior end 28 corresponding to the anterior and posterior ends 16 and 18 of the body portion 12, respectively. The attachment flange 24 is formed to extend outwardly away from the body portion 12, and it includes an adhesive thereon for securing the device 10 to the body between the legs of a human female or male.

As best shown in FIG. 5, the elongated body portion 12 is curvilinear at the posterior end 18 and has generally parallel sides as at 12a that flare laterally outwardly as at 12b to have a maximum width at the anterior end 16 thereof. The central urine collection portion 20b of the receptacle 20 also has an anterior end 30 and a posterior end 32, and the urine outlet 22 is located proximate the posterior end 32 thereof. The shallow urine directing portion 20a of the receptacle 20 includes a vent 34 which is located proximate the anterior end 16 of the body portion 12 to facilitate drainage of urine through the urine outlet 22 into a urine drain tube and drainage bag.

As shown in FIG. 5, the attachment flange 24 comprises a flat, flexible body-facing surface having the adhesive thereon and extending entirely about the perimeter of the body portion 12. The attachment flange 24 also includes a pair of anterior attachment tabs 36 and a pair of posterior attachment tabs 38 integrally associated with the attachment flange 24. Each of the attachments tabs 36 and 38 has an adhesive thereon and extends away from the body portion 12 on opposite sides of the longitudinal axis 14 to help secure the device 10 to a human female or male.

As will be appreciated, the anterior attachment tabs 36 will assist in securing the device 10 on opposite sides of the pubic region whereas the posterior tabs 38 will assist in securing the device to the buttocks.

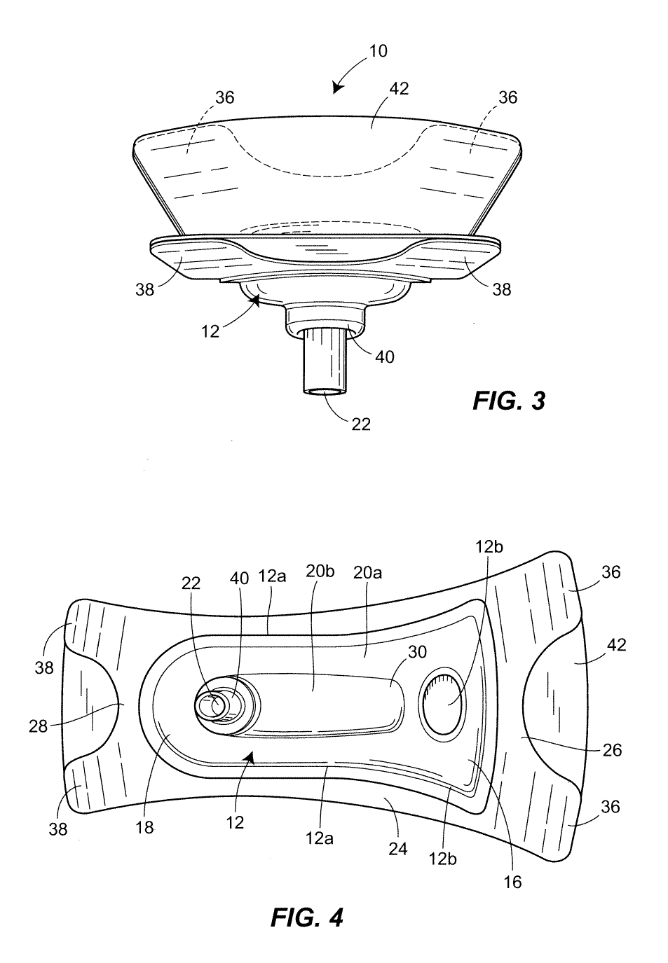

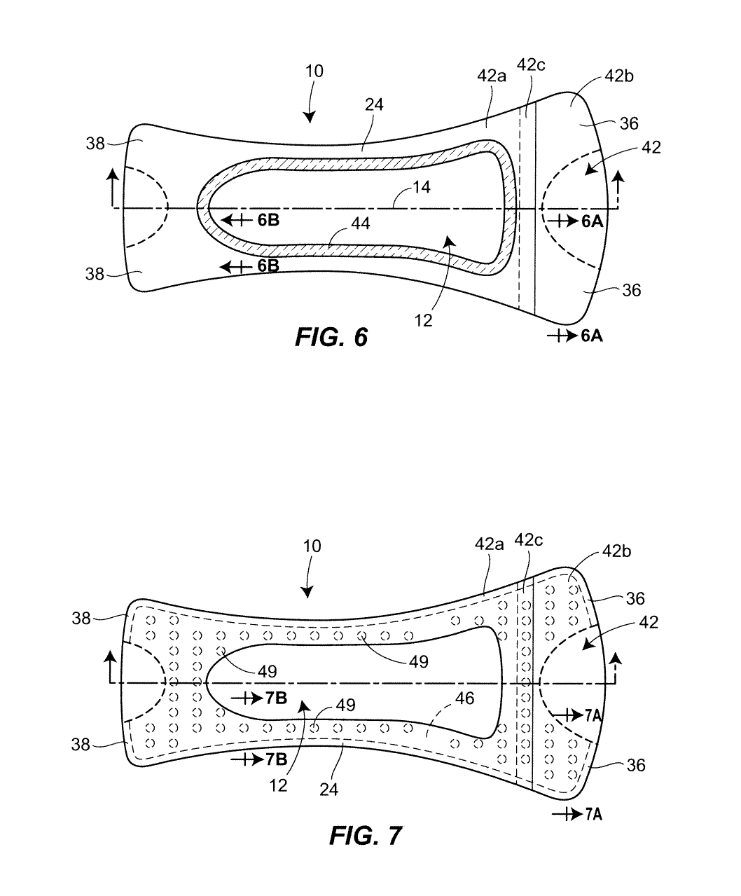

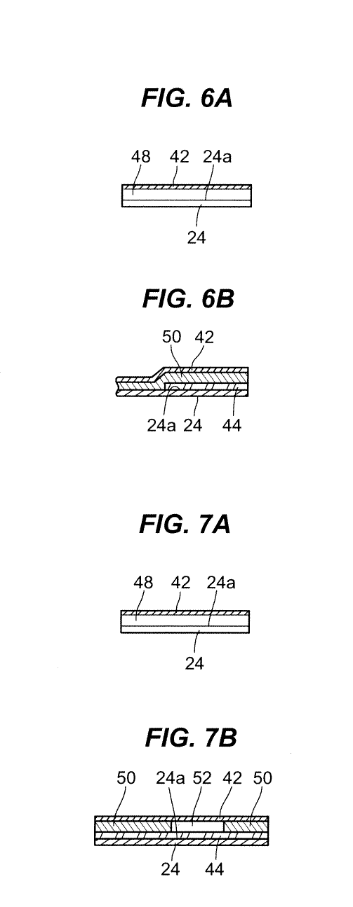

Referring to FIGS. 1-4, the urine outlet 22 may comprise a flexible tubular fitting 40 extending downwardly and rearwardly away from the central urine collection portion 20b to facilitate connection to a urine drain tube and drainage bag. The device 10 may also include a release sheet 42 (FIGS. 3 and 4) for protectively covering the adhesive on the attachment flange 24 wherein the release sheet 42 is removable to expose the adhesive for securing the device 10 to a human female covering the vulva or, alternatively, to a human male having a retracted penis to thereby cover the retracted penis. As will be seen in FIGS. 6 and 7, the release sheet 42 may comprise a split sheet having one sheet portion 42a overlapping the other sheet portion 42b as shown at 42c to facilitate gripping and removal of the sheet portions prior to using the device 10.

The device 10 may include an area of weaker adhesive strength on the anterior end 26 of the attachment flange 24 and an area of stronger adhesive strength on the remainder of the attachment flange 24. The area of weaker adhesive strength may extend from the anterior end 26 toward the posterior end 28 of the attachment flange 24 at least as far as the anterior end 16 of the body portion 12 since the anterior end will be located in the pubic region. The area of stronger adhesive strength may then extend on the remainder of the attachment flange 24 from the area of weaker adhesive strength to the posterior end 28 of the attachment flange 24.

If areas of different adhesive strength are used, the area of weaker adhesive strength will comprise the anterior tabs 36 as well as the anterior end 26 of the attachment flange 24 extending rearwardly at least to the anterior end of 16 of the body portion 12 whereas the entire remainder of the attachment flange 24 including the posterior tabs 38 will then suitably comprise the area of stronger adhesive strength. For this purpose, examples of a suitable skin-friendly adhesive for the area of weaker adhesive strength may include, but is not limited to, an elastic co-polyester film coated with a silicone gel adhesive such as BioFlex® RX1267P manufactured and sold by Scapa North America, 111 Great Pond Dr., Windsor, Conn. 06095.

The adhesive for the area of stronger adhesive strength may be made in accordance with the teaching of co-owned application US 2007/0219287, published Sep. 20, 2007, the entire disclosure of which is incorporated by reference herein. The adhesive composition comprises a network of entangled fibrillated polymeric fibers having a surface area of at least 4 m²/g, a continuous pressure-sensitive adhesive phase coating such fibers, and a discontinuous phase comprising particles of one or more liquid absorbing and swellable hydrocolloids dispersed throughout that network. The pressure-sensitive adhesive phase may be polyisobutylene (PIB) and the fibrillated fibers may be comprised of a polyolefin such as polyethylene, the latter constituting about 1% to about 5% of the total weight of the composition (hereafter referenced as wt/%). The hydrocolloids may advantageously comprise a mixture of pectin and sodium carboxymethylcellulose that may constitute about 10 to 50 wt/% of the composition. As disclosed in the aforementioned publication, variations in proportions, in the compositions of the components and their molecular weights, and other variations may occur depending on whether a given composition is intended to be used as a moldable skin barrier (of relatively high viscosity) or as a paste (of relatively low viscosity).

Specifically, an example of an adhesive composition for use in the area of stronger adhesive strength may be prepared using 55 wt./% 51,000 molecular weight PIB, 4 wt./% fibrillated polyethylene fiber (surface area of 8 m²/g, fibril length about 0.55 to 0.85 mm, fibril diameter about 15μ), 13.7 wt./% pectin, and 27.3 wt./% sodium CMC. The composition may be prepared using a Brabender Type REE6 mixer at 50° C. The ingredients may be added in the order given above, and after the addition of each ingredient, mixing is allowed to proceed until the mixture is homogeneous. After the final mixing period, the mixture is removed from the mixer and allowed to equilibrate at room conditions.

The attachment flange 24 may advantageously extend outwardly to entirely surround the body portion 12 while also extending inwardly as at 24b to define a curvilinear surface overlapping and substantially coextensive with the curvilinear posterior end 18 of the body portion 12 located posterior to the urine outlet 22 to thereby minimize the possibility of urine coming into contact with the skin as a result of pooling during maximum urine flow.

Referring to FIG. 5, the central urine collection portion 20b will be seen to have a generally oval shape. The anterior end 30 of the central urine collection portion 20b is curvilinear in shape and adjacent the vent 34 which is generally opposite the urine outlet 22 in spaced relation along the longitudinal axis 14 of the body portion 12. As shown in FIG. 5, the posterior end 32 of the central urine collection portion 20b is also curvilinear in shape.

Because of the deep, narrow elongated shape of the central urine collection portion 20b, the device 10 is believed to be capable of collecting and draining urine with little or no contact with the skin even during normal levels of maximum urine flow. As will be appreciated by referring specifically to the side elevational view of FIG. 2, the shallow urine directing portion 20a not only directs urine into the central urine collection portion 20b, but it also makes it possible for the device 10 to have a very shallow profile adding to the overall discreteness and comfort of the device 10. The only portion of the device 10 having any significant depth is the central urine collection portion 20b which will be seen to be very narrow so it will not detract from either the discreteness or the comfort of the device due to its central location.

In one illustrative example for a female urinary incontinence device, the device 10 (see FIG. 5) can have an overall length “L¹” in the direction of the longitudinal axis 14 of about 8.79″±1.30″, a length “L²” from the posterior end 18 of the body portion 12 to the anterior ends of the posterior attachment tabs 38 of about 0.65″±0.25″, a length “L³” from the anterior ends of the posterior tabs 38 to the posterior ends of the posterior tabs 38 of about 0.72″±0.25″, a length “L⁴” from the anterior end 16 to the posterior end 18 of the body portion 12 of about 5.67″±00.45″, a length “L⁵” from the anterior end 30 to the posterior end 32 of the central urine collection portion 20b of about 4.09″±0.34″, a length “L⁶” from the anterior end 16 of the body portion 12 to the posterior ends of the anterior attachment tabs 36 of about 0.62″±0.10″, a length “L⁷” from the posterior ends of the anterior attachment tabs 36 to the anterior ends of the anterior attachment tabs 36 of about 1.13″±0.25″, an overall width “W¹” at the anterior attachment tabs 36 of about 4.07″±0.50″, an overall width “W²” at the posterior attachment tabs 38 of about 2.81″±0.1″, a minimum width W³ for the attachment flange 24 of about 2.04″, a width “W⁴” for the generally parallel sides as at 12a of the elongated body portion 12 of about 1.62″±0.25″, a width “W⁵” for the laterally outwardly flared sides as at 12b of the elongated body portion 12 of about 2.77″±0.34″, a width W⁶″ for the central urine collection portion 20b of about 0.62″±0.20″, and a maximum depth “D” measured from a line drawn through the anterior and posterior ends 16 and 18 of the body portion 12 and aline tangent to the central urine collection portion 20b of approximately 1.19″±0.25″ (see FIG. 2).

In one illustrative example for a male urinary incontinence device, the device 10 (see FIG. 5) can have an overall length “L¹” in the direction of the longitudinal axis 14 of about 8.94″±1.45″, a length “L²” from the posterior end 18 of the body portion 12 to the anterior ends of the posterior attachment tabs 38 of about 1.04″±0.25″, a length “L³” from the anterior ends of the posterior tabs 38 to the posterior ends of the posterior tabs 38 of about 0.59″±0.25″, a length “L⁴” from the anterior end 16 to the posterior end 18 of the body portion 12 of about 5.67″±0.45″, a length “L⁵” from the anterior end 30 to the posterior end 32 of the central urine collection portion 20b of about 4.09″±0.34″, a length “L⁶” from the anterior end 16 of the body portion 12 to the posterior ends of the anterior attachment tabs 36 of about 0.60″±0.15″, a length “L⁷” from the posterior ends of the anterior attachment tabs 36 to the anterior ends of the anterior attachment tabs 36 of about 1.04″±0.35″, an overall width “W¹” at the anterior attachment tabs 36 of about 4.07″±0.50″, an overall width “W²” at the posterior attachment tabs 38 of about 2.81″±0.1″, a minimum width W³ for the attachment flange 24 of about 2.27″, a width “W⁴” for the generally parallel sides as at 12a of the elongated body portion 12 of about 2.35″±0.75″, a width “W⁵” for the laterally outwardly flared sides as at 12b of the elongated body portion 12 of about 2.85″±0.40″, a width W⁶” for the central urine collection portion 20b of about 0.62″±0.20″, and a maximum depth “D” measured from a line drawn through the anterior and posterior ends 16 and 18 of the body portion 12 and a line tangent to the central urine collection portion 20b of approximately 1.95″±0.50″ (see FIG. 2).

With regard to the length, width and depth dimensional ratios between the shallow urine directing portion 20a and the deeper, narrower urine collecting portion 20b, the length ratio may suitably comprise about 1.4:1, the width ratio may suitably comprise about 1.67:1 and the depth ratio may suitably comprise about 1.25:1.

As an additional feature, the device 10 may include absorptive foam on some or substantially all of the attachment flange 24 (see FIGS. 6 and 7). Referring to FIG. 6, the attachment flange 24 is provided with a bead of absorptive foam as at 44 about the perimeter of the body portion 12 whereas substantially the entirety of the attachment flange 24 including the anterior and posterior attachment tabs 36 and 38 is provided with absorptive foam as at 46 in the embodiment illustrated in FIG. 7. In both embodiments, the absorptive foam is applied directly to the attachment flange 24 and the adhesive is then applied over the absorptive foam.

Referring to FIGS. 6A and 6B, the details of the materials used in the embodiment of the device 10 illustrated in FIG. 6 at the indicated locations of 6A-6A and 6B-6B, respectively, can be understood. It will be seen from FIG. 6A that the attachment flange 24 preferably has the weaker adhesive layer 48 applied directly to the body facing surface 24a wherein the adhesive layer 48 is covered by a protective release sheet 42 whereas, in FIG. 6B, it will be seen that the area of absorptive foam 44 is disposed on the body facing surface 24a. With this arrangement, it will be understood that the stronger adhesive layer 50 used in this portion of the device 10 is applied directly to the body facing surface 24a except where the absorptive foam 44 is located.

In this area, the stronger adhesive layer 50 extends over the absorptive foam 44 so that the absorptive foam is located between the body facing surface 24a of the attachment flange 24 and the stronger adhesive layer 50 which is covered by the protective release sheet 42.

Referring to FIGS. 7A and 7B, the details of the materials used in the embodiment of the device 10 illustrated in FIG. 7 at the indicated locations of 7A-7A and 7B-7B, respectively, can be understood. It will be seen from FIG. 7A that the attachment flange 24 preferably has the weaker adhesive layer 48 applied directly to the body facing surface 24a wherein the adhesive layer 48 is covered by a protective release sheet 42 whereas FIG. 7 shows the area of absorptive foam 44 disposed on substantially the entire body facing surface 24a . With this arrangement, the stronger adhesive layer 50 used in the location 7B-7B of the device 10 is applied directly over the absorptive foam 44 covering the body facing surface 24a of the attachment flange 24.

In this area, the stronger adhesive layer 50 extends over the absorptive foam 44 to form an adhesive pattern having holes 52 therein to expose the absorptive foam 44 during use of the device 10, and the absorptive foam 44 is located between the body facing surface 24a of the attachment flange 24 and patterned stronger adhesive layer 50 which is covered by the protective release sheet 42.

In the embodiment illustrated in FIG. 7, a pattern coat of the adhesive is applied over the absorptive foam 46 to provide a regular pattern of holes 49 to expose the foam, and in both of the embodiments illustrated in FIGS. 6 and 7 the release sheet 42 is applied over the adhesive to cover not only the adhesive but also the absorptive foam.

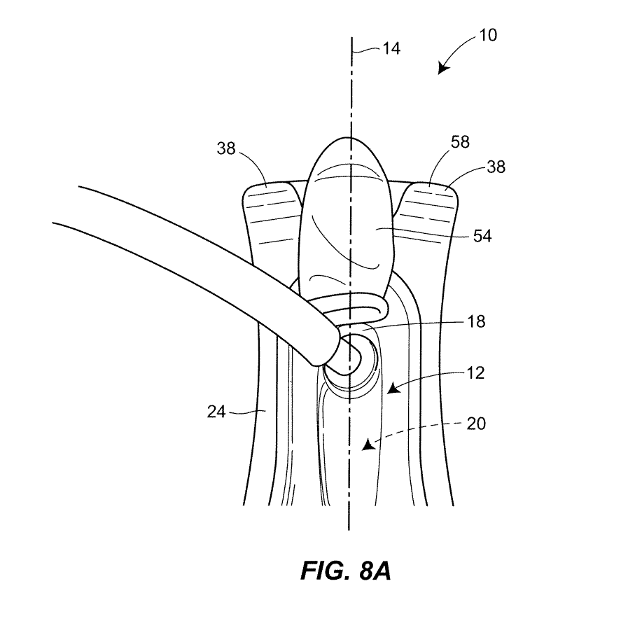

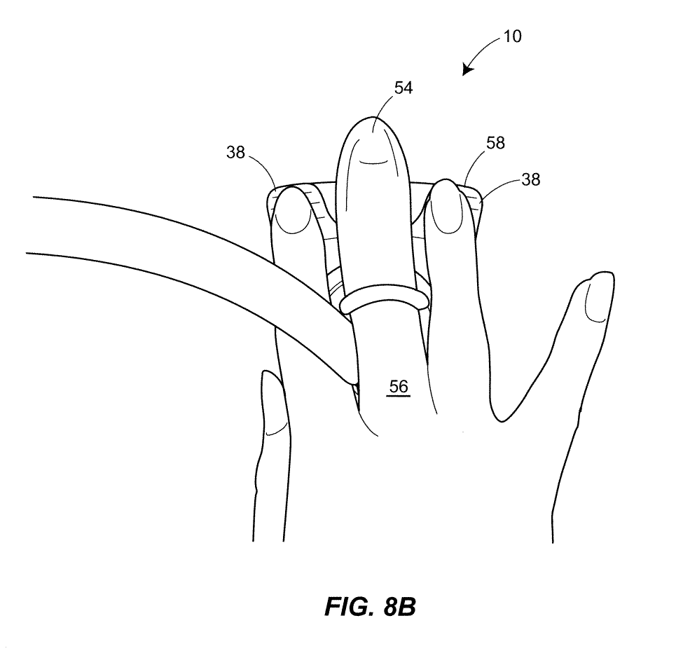

Referring to FIGS. 8A and 8B, a method for positioning a urinary incontinence device such as 10 can be understood as comprising the step of providing a flexible but shape-retaining body portion such as 12. The body portion 12 will be understood to have an anterior end (not shown) and a posterior end such as 18 and to define a receptacle such as 20 for receiving urine where the receptacle is surrounded by a flange such as 24. The flange 24 has an adhesive thereon for securing the device 10 in a position for use with the body portion 12 facing the vulva or retracted penis of a human female or male. The method also includes the step of providing a finger shield such as 54 on the posterior end of the body portion 12 externally of the urine-receiving receptacle 20. The method also includes the step of placing a finger such as 56 in the finger shield 54 and positioning the device 10 with the urine-receiving receptacle 20 facing the vulva or retracted penis of a human female or male. The method also includes the step of locating the anus with the finger 56 in the finger shield 54 before the adhesive on the flange 24 makes substantial contact with the skin of the human female or male. Further, the method includes the step of applying pressure to the flange 24 to cause the adhesive to make substantial contact with the skin to secure the device 10 to the human female or male in a position for use.

In one embodiment, the method may include the step of removing the finger shield 54 from the posterior end 18 of the body portion 12 after the device has been secured to a human female or male in a position for use. The method may also include the flange 24 having a posterior end 58, in this case the posterior end of the tabs 38, and the finger shield 54 may extend approximately 1 inch beyond the posterior end 58 of the flange 24. In still another embodiment, the method may include the body portion 12 having a longitudinal axis such as 14 and the finger shield 54 being located to lie along the longitudinal axis 14 of the body portion 12.

While in the foregoing exemplary embodiments of the disclosure have been set forth, it will be understood that the details herein given may be varied by those skilled in the art without departing from the true scope and spirit of the appended claims.

What is claimed is:

1. A urinary incontinence device, comprising:
a flexible but shape-retaining body portion having a longitudinal axis extending from an anterior end to a posterior end of the body portion, the body portion being elongated in the direction of the longitudinal axis, the body portion having a length and a width relative to the longitudinal axis sufficient to define a receptacle for receiving urine when the device is placed on a body of a human female or male in a position for use;
the urine-receiving receptacle having a shallow urine directing portion sloping inwardly and downwardly away from a body in a position for use, the urine-receiving receptacle also having a deeper, narrower central urine collection portion elongated in the direction of the longitudinal axis of the body portion, the central urine collection portion having a urine outlet for urine to drain from the body portion away from a body in a position for use; and
an attachment flange having an anterior end and a posterior end corresponding to the anterior end and the posterior end of the body portion, the attachment flange extending outwardly of the body portion, the attachment flange having an adhesive thereon for securing the device in a position to cover a vulva or retracted penis of a human female or male.

2. The urinary incontinence device of claim 1 wherein the elongated body portion is curvilinear at the posterior end and has generally parallel sides that flare laterally outwardly to have a maximum width at the anterior end.

3. The urinary incontinence device of claim 1 wherein the central urine collection portion of the receptacle has an anterior end and a posterior end and the urine outlet is located proximate the posterior end thereof.

4. The urinary incontinence device of claim 1 wherein the shallow urine directing portion of the receptacle includes a vent located proximate the anterior end of the body portion to facilitate drainage through the urine outlet.

5. The urinary incontinence device of claim 1 wherein the attachment flange comprises a flat, flexible body-facing surface having the adhesive thereon and extending entirely about the perimeter of the body portion.

6. The urinary incontinence device of claim 5 including a pair of anterior attachment tabs and a pair of posterior attachment tabs each integrally associated with the attachment flange on opposite sides of the longitudinal axis.

7. The urinary incontinence device of claim 6 wherein each of the attachment tabs has an adhesive thereon and extends away from the body portion to assist the attachment flange in securing the device to a human female or male.

8. The urinary incontinence device of claim 1 wherein the urine outlet includes a flexible tubular fitting extending downwardly and rearwardly away from the central urine collection portion for connection to a urine drain tube and drainage bag.

9. The urinary incontinence device of claim 1 including a release sheet for protectively covering the adhesive on the attachment flange but removable to expose the adhesive for securing the device to a human female or male.

10. The urinary incontinence device of claim 1 including an area of weaker adhesive strength on the anterior end of the attachment flange and an area of stronger adhesive strength on the remainder of the attachment flange.

11. The urinary incontinence device of claim 10 wherein the area of weaker adhesive strength extends from the anterior end toward the posterior end of the attachment flange at least as far as the anterior end of the body portion.

12. The urinary incontinence device of claim 11 wherein the area of stronger adhesive strength extends on the remainder of the attachment flange from the area of weaker adhesive strength to the posterior end of the attachment flange.

13. The urinary incontinence device of claim 1 wherein the attachment flange extends outwardly to entirely surround the body portion and extends inwardly over the posterior end of the body portion posterior toward the urine outlet.

14. The urinary incontinence device of claim 1 including an absorptive foam on at least a portion of the attachment flange, the absorptive foam being disposed between the attachment flange and the adhesive.

15. The urinary incontinence device of claim 14 including a regular pattern of holes in the adhesive to expose the absorptive foam.

16. The urinary incontinence device of claim 1 having a finger shield associated with the posterior end of the body portion externally of the urine-receiving receptacle for placing the device in a position for use.

17. A urinary incontinence device, comprising:
a flexible but shape-retaining elongated body portion adapted to entirely cover the vulva or retracted penis of a human female or male, the body portion having a longitudinal axis extending from an anterior end to a posterior end thereof;
the body portion forming a urine-receiving receptacle when positioned to cover the vulva or retracted penis of a human female or male, the receptacle including a shallow urine directing portion sloped inwardly and downwardly away from the vulva or retracted penis, the receptacle also including a deeper, narrower elongated central urine collection portion disposed along the longitudinal axis of the body portion, the central urine collection portion of the receptacle having a urine outlet for urine to drain from the body portion;
the central urine collection portion of the receptacle having an anterior end and a posterior end with the urine outlet being located proximate the posterior end;
a vent located proximate the anterior end of the body portion in the shallow urine directing portion of the receptacle formed by the body portion so as to be generally opposite the urine outlet in spaced relation along the longitudinal axis of the body portion; and
an attachment flange extending outwardly of the body portion and having an anterior end and a posterior end corresponding to the anterior end and the posterior end of the body portion, the attachment flange having an adhesive thereon for securing the device in a position such that the body portion entirely covers the vulva or retracted penis of a human female or male.

18. The urinary incontinence device of claim 17 wherein the elongated body portion is curvilinear at the posterior end and has generally parallel sides that flare laterally outwardly to have a maximum width at the anterior end.

19. The urinary incontinence device of claim 17 wherein the attachment flange comprises a flat, flexible body-facing surface having the adhesive thereon and extending about the perimeter of the body portion, the attachment flange having a pair of anterior attachment tabs and a pair of posterior attachment tabs each integrally associated therewith on opposite sides of the longitudinal axis, each of the attachment tabs having an adhesive thereon and extending away from the body portion to assist the attachment flange in securing the device to entirely cover the vulva or retracted penis of a human female or male.

20. The urinary incontinence device of claim 17 including a first adhesive on the anterior end of the attachment flange and a second adhesive on the remainder of the attachment flange, the first adhesive defining an area of weaker adhesive strength and extending from the anterior end of the attachment flange toward the posterior end thereof so as to be at least proximate the anterior end of the body portion, the second adhesive defining an area of stronger adhesive strength extending on the remainder of the attachment flange from the area of weaker adhesive strength to the posterior end of the attachment flange.

21. The urinary incontinence device of claim 17 wherein the urine outlet includes a flexible tubular fitting extending downwardly and rearwardly away from the central urine collection portion for connection to a urine drain tube and drainage bag.

22. The urinary incontinence device of claim 17 including a release sheet for protectively covering the adhesive on the attachment flange but removable to expose the adhesive for securing the device to a human female or male.

23. The urinary incontinence device of claim 17 wherein the attachment flange extends outwardly to entirely surround the body portion and extends inwardly over the posterior end of the body portion posterior toward the urine outlet.

24. The urinary incontinence device of claim 17 including an absorptive foam on at least a portion of the attachment flange, the absorptive foam being disposed between the attachment flange and the adhesive.

25. The urinary incontinence device of claim 24 including a regular pattern of holes in the adhesive to expose the absorptive foam.

26. The urinary incontinence device of claim 17 having a finger shield associated with the posterior end of the body portion externally of the urine-receiving receptacle for placing the device in a position for use.

27. A urinary incontinence device, comprising:
a flexible but shape-retaining elongated body portion adapted to entirely cover the vulva or retracted penis of a human female or male, the body portion having a longitudinal axis extending from an anterior end to a posterior end thereof, the body portion being flared laterally outwardly to have a maximum width at the anterior end thereof;
the body portion forming a urine-receiving receptacle when positioned to cover the vulva or retracted penis of a human female or male, the receptacle including a shallow urine directing portion sloped inwardly and downwardly away from the vulva or retracted penis and a deeper, narrower elongated central urine collection portion disposed along the longitudinal axis of the body portion, the central urine collection portion of the receptacle having a urine outlet for urine to drain from the body portion;
the central urine collection portion of the receptacle having an anterior end and a posterior end with the urine outlet being located proximate the posterior end and having a flexible tubular fitting extending downwardly and rearwardly away from the central urine collection portion for connection to a urine drain tube and drainage bag;
a vent located proximate the anterior end of the body portion in the shallow urine directing portion of the receptacle formed by the body portion so as to be generally opposite the urine outlet in spaced relation along the longitudinal axis of the body portion; and
an attachment flange having an anterior end and a posterior end corresponding to the anterior end and the posterior end of the body portion, the attachment flange extending outwardly to surround the body portion and extending inwardly over the posterior end of the body portion toward the urine outlet, the attachment flange having an adhesive thereon for securing the device to entirely cover the vulva or retracted penis of a human female or male.

28. The urinary incontinence device of claim 27 wherein the elongated body portion is curvilinear at the posterior end, the inwardly extending portion of the attachment flange defining a curvilinear surface overlapping and substantially coextensive with the curvilinear posterior end of the body portion, the body portion having generally parallel sides that flare laterally outwardly to have a maximum width at the anterior end.

29. The urinary incontinence device of claim 27 wherein the attachment flange comprises a flat, flexible body-facing surface having the adhesive thereon and extending about the perimeter of the body portion, the attachment flange having a pair of anterior attachment tabs and a pair of posterior attachment tabs each integrally associated therewith on opposite sides of the longitudinal axis, each of the attachment tabs having an adhesive thereon and extending away from the body portion to assist the attachment flange in securing the device to entirely cover the vulva or retracted penis of a human female or male.

30. The urinary incontinence device of claim 27 including a first adhesive on the anterior end of the attachment flange and a second adhesive on the remainder of the attachment flange, the first adhesive defining an area of weaker adhesive strength and extending from the anterior end of the attachment flange toward the posterior end thereof so as to be at least proximate the anterior end of the body portion, the second adhesive defining an area of stronger adhesive strength extending on the remainder of the attachment flange from the area of weaker adhesive strength to the posterior end of the attachment flange.

31. The urinary incontinence device of claim 27 including a release sheet for protectively covering the adhesive on the attachment flange but removable to expose the adhesive for securing the device to a human female or male.

32. The urinary incontinence device of claim 27 including an absorptive foam on at least a portion of the attachment flange, the absorptive foam being disposed between the attachment flange and the adhesive.

33. The urinary incontinence device of claim 32 including a regular pattern of holes in the adhesive to expose the absorptive foam.

34. The urinary incontinence device of claim 27 having a finger shield associated with the posterior end of the body portion externally of the urine-receiving receptacle for placing the device in a position for use.

35. A method for positioning a urinary incontinence device, comprising the steps of:
providing a flexible but shape-retaining body portion having an anterior end and a posterior end and defining a receptacle for receiving urine surrounded by a flange having an adhesive for securing the device to a human female or male in a position for use;
providing a finger shield on the posterior end of the body portion externally of the urine-receiving receptacle for use in locating the device in a position for use;
placing a finger in the finger shield and positioning the device with the urine-receiving receptacle facing the vulva or retracted penis of a human female or male;
locating the anus with the finger in the finger shield before the adhesive on the flange makes any substantial contact with the skin of the human female or male; and
applying pressure to the flange to cause the adhesive to make substantial contact with the skin to secure the device to a human female or male in a position for use.

36. The method of claim 35 including the step of removing the finger shield from the posterior end of the body portion after the device has been secured to a human female or male in a position for use.

37. The method of claim 35 wherein the flange has a posterior end and the finger shield extends approximately 1 inch beyond the posterior end of the flange.

38. The method of claim 35 wherein the body portion has a longitudinal axis and the finger shield is located to lie along the longitudinal axis of the body portion.
